(19) United States
(12) Patent Application Publication
(10) Pub. No.: US 2006/0067899 A1
(43) Pub. Date: Mar. 30, 2006

(54) Paint for skin

(75) Inventor: Yukiya Tanemo, Tokyo (JP)

Correspondence Address: GREENBLUM & BERNSTEIN, P.L.C., 1950 ROLAND CLARKE PLACE, RESTON, VA, 20191, US

(73) Assignee: Hagalex Japan Co., Ltd., Tokyo (JP)

(21) Appl. No.: 10/952,775

(22) Filed: Sep. 30, 2004

(51) Int. Cl. A61K 8/81

(52) U.S. Cl. 424/63

(57) ABSTRACT

A paint for skin which can eliminate the possibility that the paint will drip off or that there will blurring or mixing of colors with rain or sweat when the paint is used for a face paint or body paint, and which can be removed easily and completely only with fingertips without washing with water or a soap after use, the paint being prepared by blending a rubber latex which forms a peelable rubber film on a skin surface when applied to the human skin, with a viscosity improver for providing a viscosity to the rubber latex to such an extent that sagging does not occur upon application on the skin and a pigment for applying a color to the skin.

### BACKGROUND OF THE INVENTION

1. Field of the Invention

The present invention concerns a paint for skin preferably used for a face paint, body paint or makeup.

2. Statement of the Related Art

There are aqueous paints for makeup and oily paints using a color ink for surgery marking, as commercially available paints for skin. They are used, for example, for a body paint with which colorful patterns are drawn on an arm, or for a face paint with which supporters cheering a soccer team or spectators viewing a soccer game draw on their faces a flag of a team they support or a national flag of their country.

The aqueous paint for makeup is used for drawing a desired pattern on a skin surface of a face or arm with a fingertip or a cotton-tipped swab, which is dried with body heat. However, in a case where it is wetted with rain or sweat, blurs or mixing of colors occurs in the pattern drawn with the paint, the paint drips off to lose its shape or the pattern sometimes disappears.

Further, there has been a possibility that the paint drips off with rain or sweat and adheres to and stains the clothes. Moreover, in order to remove the paint from the skin surface completely after use, a troublesome procedure is required, entailing washing it with water or wiping it off using a cloth wetted with water or wet tissue.

On the other hand, while an oily paint using a marking ink for surgery has an advantage that it does not drip off during the use when wetted with rain or sweat, it requires a troublesome procedure to remove it by using a soap or cleansing foam after use. In addition, the marking ink for surgery sometimes causes allergic itch or anthema when it is applied on the skin.

### SUMMARY OF THE INVENTION

Then, the present invention intends to provide a paint for skin which causes no blur or mixing of colors when wetted with rain or sweat during use, and will not drip off while wetted with rain or sweat, and moreover, which can be removed easily and completely after use without washing with water, a soap or a cleansing foam.

In order to solve the problems described above, the present invention provides a paint for skin prepared by blending a rubber latex which forms a peelable rubber film on the human skin surface when applied on a human skin, with a viscosity improver for increasing the viscosity of the rubber latex to such an extent that sagging does not occur upon application thereof and a pigment for applying a color to the skin.

Since the paint for skin has a viscosity of such an extent that sagging does not occur when applied on a human skin, a desired pattern can be drawn on the skin surface with a fingertip or brush for makeup. Further, when it is dried, a rubber film adhering on the skin surface is formed. Accordingly, there is no possibility of causing blurs, mixing of colors, or dripping off of the rubber film when wetted with rain or sweat. The rubber film can be removed from the skin surface easily and completely after use only by picking up the edge of the rubber film adhering on the skin surface with fingertips.

### DESCRIPTION OF THE PREFERRED EMBODIMENT

Preferred embodiments of the present invention will be explained below.

The paint for skin according to the invention comprises a rubber latex, as a main ingredient, which forms a peelable rubber film when applied on a human skin, blended with a viscosity improver for increasing the viscosity of the rubber latex to such an extent that sagging does not occur upon application thereof and a pigment for applying a color to the skin.

As the rubber latex, natural latexes which are gentle to skin are preferred, especially, a deproteinized latex which is prepared by removing protein, a substance causing hypersensitive reaction, from natural rubber components is optimal for the paint for skin since it is a safe substance that is registered as a cosmetic, so that even persons having sensitive skin can use it without worry.

As the thickening agent, hydroxypropyl methyl cellulose is optimal, since also is a safe substance that is registered as a cosmetic, and since it has good affinity with natural latexes.

Further, as the pigment, it is preferred to use organic pigments or inorganic pigments such as a ceramic color powder that are registered as cosmetics.

An optimal embodiment is prepared by blending 100 parts by weight of a deproteinized latex (“SELATEX” manufactured by Sumitomo Rubber Industry Co. Ltd.) with from to 10 parts by weight of hydroxypropyl methyl cellulose as a viscosity improver (“Metlose 10% solution”, manufactured by Shin-Etsu Chemical Co. Ltd.), which is excellent in effectiveness and safety as a paint for skin.

In other words, both the deproteinized latex and the hydroxypropyl methyl cellulose as a viscosity improver are safe substances that are registered as cosmetics, and there is no possibility that they will cause allergy when applied to skin.

It was confirmed by experiments that a desired pattern could be drawn with the paint prepared by blending the deproteinized latex and the viscosity improver at the ratio described above, it could be spread smoothly with a fingertip or a cotton-tipped swab with no sagging when applied on the skin.

Further, the paint having the blending ratio described above dries in about 2 min after the application to the skin, and forms a rubber film firmly adhering on the skin surface. Since the rubber film has elasticity and toughness of such an extent that it does no tear when it is picked up at one edge so as to be peeled off from the skin surface with fingertips, the paint can be peeled off from the skin surface quickly and removed completely with ease.

Further, in a case where the blending amount of the viscosity improver is decreased to less than 5 parts by weight, the fluidity of the rubber latex is increased, tending to cause sagging upon application to the skin, to result in a problem that it is difficult to draw a desired pattern with a fingertip or a brush for makeup.

On the contrary, in a case where the blending amount of the viscosity improver is increased to more than 10 parts by weight, the viscosity becomes excessively large, so that it is difficult to spread the paint applied on the skin smoothly on the surface with a fingertip or the like and the drying rate of the paint is increased, causing the problem that it is impossible to draw a desired pattern, and also causing the problem that the rubber film formed on the skin surface tends to tear due to its brittleness. Accordingly, it is impossible to peel off the rubber film completely intact from the skin surface by picking an edge with fingertips.

Then, with respect to the pigment for applying a color to the skin, organic pigments which are also registered as cosmetics are used, and the blending ratio to provide coloration to such an extent that the skin texture is not seen through the paint upon application on the skin is in a range from about 2 to 20 parts by weight based on 100 parts by weight of the deproteinized latex although it is different depending on the color and the material of each pigment.

In addition, ammonia is applied as a preservative in the deproteinized latex. In order to mitigate the odor of the ammonium, the deproteinized latex is subjected to heat treatment at 60° C. and stirred for 24 hours. Accordingly, there is no possibility that acrid or unpleasant odors will be generated when the cosmetic paint is applied on the face.

The description above is one example of the paint for skin according to the invention, and the method of use and functional effects thereof are to be explained next.

The paints for skin of the invention are filled each in a tightly sealed tube with a lid in the same manner as general watercolors or oil colors, and used while being squeezed out in each appropriate amount from the opening of each tube to a fingertip or a brush for makeup.

Then, the paint squeezed out to the fingertip is applied directly on the skin with the fingertip and a desired pattern is drawn quickly by spreading the paint on the skin surface. The paint thus applied on the skin dries with body heat to form a peelable rubber film adhering on the skin surface. Since the rubber film has water resistance, there is no possibility of causing mixing of pigment colors or blurs even if it is wetted with rain or sweat, or of dripping off with rain or sweat. Accordingly, the shape of the pattern drawn with the paint will not be lost and the pattern will not disappear, as well as there is no possibility of staining clothes.

Further, since the paint, which is in the form of a rubber film, adhering on the skin surface can be peeled off from the skin surface easily and completely by picking up an edge of the rubber film with fingertips, no troublesome procedure to wash it with water or a detergent is required after use.

As described above, since the paint for skin according to the invention causes less sagging when applied to the skin, a desired pattern can be drawn on the skin surface. Further, when the paint is applied on the skin and dried, a water-resistant rubber film is formed, and accordingly, the pattern drawn with the paint neither causes blurs or mixing of colors when wetted with rain or sweat nor drips off with rain or sweat, loses its shape or disappears. Further, the rubber film adhering on the skin can be removed easily and completely after use merely by picking up an edge of the rubber film adhering on the skin surface with fingertips.

1. A paint for skin prepared by blending a rubber latex which forms a peelable rubber film when applied to the skin surface, with a viscosity improver for providing a viscosity to the rubber latex to such an extent that sagging does not occur upon application on the skin, and a pigment for applying a color to the skin.

2. A paint for skin according to claim 1, wherein the rubber latex is a deproteinized latex prepared by removing a protein from a natural rubber component.

3. A paint for skin according to claim 2, wherein from 5 to 10 parts by weight of the viscosity improver is blended based on 100 parts by weight of the deproteinized latex.

4. A paint for skin according to claim 2, wherein ammonia is added as a preservative to the deproteinized latex and heat treatment is conducted to mitigate the odor of ammonia.

5. A paint for skin according to claim 3, wherein ammonia is added as a preservative to the deproteinized latex and heat treatment is conducted to mitigate the odor of ammonia.
